(19) United States
(12) Patent Application Publication
(10) Pub. No.: US 2004/0115258 A1
(43) Pub. Date: Jun. 17, 2004

(54) Oral pharmaceutical compositions containing cyclodextrins as taste masking agent

(76) Inventors: Federico Stroppolo, Aldesago (CH); Franco Ciccarello, Mezzovico (CH); Rita Milani, Cannobio (CH); Lorenzo Bellorini, Monate (IT)

Correspondence Address: Synnestvedt & Lechner, 2600 Aramark Tower, 1101 Market Street, Philadelphia, PA, 19107-2950, US

(21) Appl. No.: 10/432,575

(22) PCT Filed: Nov. 23, 2001
(86) PCT No.: PCT/GB01/05212

(30) Foreign Application Priority Data
Nov. 23, 2000 (GB) 0028575.9

(51) Int. Cl. A61K 31/724; A61K 9/20

(52) U.S. Cl. 424/465; 514/58

(57) ABSTRACT

The application discloses oral pharmaceutical compositions which are tasted in the mouth during administration. Fast-dissolving tablets, chewable tablets and effervescent dispersions are exemplified. To mask the taste of unpleasant-tasting active ingredients, it has been found that blending with cyclodextrin without the conventional complex formation is effective. Consequently more economical modes of manufacture such as simple granulation and dry blending can be used.

### FIELD OF THE INVENTION

The present invention relates to the use of cyclodextrin for taste-masking in orally administered pharmaceutical compositions, to the pharmaceutical compositions themselves and to processes for making them.

### BACKGROUND

Cyclodextrins are cyclic oligosaccharides formed from α-(1,4)-linked D-glucopyranose units. α, βand γ-cyclodextrins consist of six, seven and eight units respectively. The molecules have a toroidal shape, with a hydrophobic central cavity and a relatively hydrophilic outer surface. This structure enables cyclodextrins to bind appropriately sized non-polar guest molecules, or moieties of guest molecules, within the hydrophobic central cavity, to form clathrate complexes. Because the exterior of the cyclodextrin is relatively hydrophilic, formation of such complexes may therefore be used to increase the solubility of otherwise poorly soluble molecules.

There has long been an interest in cyclodextrins in the pharmaceutical industry. They have been used to increase the solubility, stability and bioavailability of a variety of active drug molecules in drug formulations, and also to mask the taste of certain active ingredients, by exploiting this formation of complexes between the active ingredient and the cyclodextrin.

There is currently an increasing demand for orally administrable formulations of pharmaceuticals, because of good associated patient compliance. However, conventional solid tablet formulations which are swallowed whole are often not ideal for administration of active ingredients. Many patients, especially the very young or old, find it difficult to swallow tablets whole, and bioavailability of the active ingredient can be poor. More preferable therefore are effervescent or other soluble formulations which can be dissolved and drunk, chewable or fast melting tablets, and slow release formulations such as sub-lingual tablets, which are placed under the tongue and enable absorption of the active ingredient into the bloodstream through the oral mucosa. Such formulations may reduce the time taken for drugs to be taken up and begin to act, and increase the bioavailability of the drug. However patient compliance may be low when the active ingredients have a markedly unpleasant taste.

What is described here generally as “unpleasant taste” may be any of for example a bitter taste, burning taste, salty taste or other generally revolting taste. It is well-known in the pharmaceutical field that some active ingredients taste so severely unpleasant that patient compliance in a tasted oral formulation is out of the question unless the taste can be masked.

As mentioned above, it is known that drug palatability can be improved by formation of cyclodextrin inclusion complexes of the unpalatable active ingredient. See for example U.S. Pat. No. 5,206,025 describing special freeze-dried oral formulations of cyclodextrin complexes of active ingredient, designed to disintegrate rapidly in the mouth and with masking of unpleasant-tasting active ingredient.

### SUMMARY OF THE INVENTION

The present invention is based on the wholly unexpected finding that cyclodextrin can be effective to mask the taste of pharmaceutically active agents without the formulation of inclusion complexes between the cyclodextrin and the active agent, a step conventionally thought to be essential. This has important implications in terms of both products and production processes as regards simplicity and economy.

Thus, in one aspect the present invention provides use of a cyclodextrin to mask the taste, and particularly the unpleasant taste, of an active ingredient in a pharmaceutical preparation adapted for oral administration, wherein the active ingredient in the preparation is substantially uncomplexed by the cyclodextrin.

A second aspect is a process of making a solid oral pharmaceutical preparation which includes blending the active ingredient—and particularly one that has an unpleasant taste—with cyclodextrin under conditions which do not promote complex formation between the cyclodextrin and active ingredient.

A further aspect of the invention is the use, in the oral administration of a solid pharmaceutical preparation of cyclodextrin blended but not complexed with active ingredient in the preparation to mask the taste of the uncomplexed active ingredient.

A further aspect is the use, in the preparation of a solid oral pharmaceutical preparation of the kind described, of cyclodextrin as a taste-masking agent for uncomplexed active ingredient contained in the preparation. These and other aspects of the invention are set out in the claims.

As regards the nature of the active ingredient, the invention may have use in any situation in which it has a taste which is sought to be masked, and in particular when this is an unpleasant or very unpleasant taste.

We have noted that in the prior art EP 0 839 528 A (Staroil Ltd.) dicloses use of β-cyclodextrin in mouth-soluble N-acetylcysteine compositions, in order to mask (by complexing) the taste of a sulphated degradation product of N-acetylcysteine. Thus the cyclodextrin was disclosed for use to mask the taste not of the active ingredient, but of a degradation product formed during storage of the composition. Thus, the present proposals may not extend to preparations of kind described in which the active ingredient is N-acetylcysteine.

However, in general the invention may be used with a wide variety of active ingredients. A non-exhaustive list of active ingredients whose taste could usefully by improved or masked include the following.

Examples not limited of categories of actives that could be improved from taste point of view are:

Abortifacients e.g. Prostaglandin E2, Mifepristone

ACE Inhibitors, e.g. Benazepril, Captopril, Delapril, Ena1april, Imidapril, Ramipril:

α-Adrenergic Agonists e.g. Adrenolone, Clonidine, Ephedrine, Epinephrine, Phenylephrine, Fenoxazoline, Ibopamine, Methoxamine, Nafazoline, Pseudoephedrine, Tetrahydrozoline, Tramazoline, Phenyilpropanolamine, Tuaminoheptane, Tyramine Xylomethazoline

β-Adrenergic AgonistS˜.: Albuterol, bambuterol, Clenbuterol, Clorprenaline, Dopexamine, Ephedrine, Epinephrine, Ethylnorepinephrine, F enoterolo, F ormoterolo, Isoproterenolo, Mabuterolo, Metaproterenolo, Methoxiphenamine, Oxyfedrine, Reproterolo, Salmeterol, Soterenol, Terbuta1ine, Tulobuterol, Xanoterol

α Adrenergic Blockers e.g. Dapiprazole, Fenspiride, Nicergoline, Prazosin, Yohimbine,

β-Adrenergic Blockers e.g.: Acebutolol, A1prenolol, Atenolol, Befnolol, Betaxolol, Bpindolol, Bupranolol, Carazolol, Carteolol, Celiprolol, Indenolol, Levobunolol, Mepindolol, Metipranolol, Moprolol, Pindolol, Practolol, Propranolol, Timolol;

Adrenocortical Steroids

Adrenocorticotrop Hormones e.g. ACTH Cosintropin

Alcohol deterrents e.g. Calcium lanamide Citrate, Disulfiram

Aldose reductase inhibitors e.g. Epalrestat, Tolrestat, Zopolrestati

Aldosterone Antagonists e.g. Canrenone, Spironolattone;.

Anabolics e.g. Androisoxazole, Androstenediol, Methandriol, Methenolon, Methiltrienolone, Nandrolone;

Analgesics, (Narcotic), e.g. Alfentanil, Buprenorphone, Codine and its derivatives, Fentanil, Meperidine, Methadone, Morphine and its derviatives Phenazocine, Propiram, Propoxiphene, Sufentanil:

Analgesict (Non Narcotic) e.g. Aceclofenac, Acetaminophen, Acetysalicyic acid, Alclofenac, Alminoprofen, Antypirine, Benorilate, Benoxoprofen, Bromfenac, Bucetin, Carbamazepine, Carbiphene, Ch1ortenoxazin, Cholin salicyate, Clometacin, Clonixin, Croropamide, Diflunisa1, Etodolac, Felbinac, Fenoprofen, Flufenamiv acid, Flurbiprofen, Ibufenac, Imidazole salicylate, Indomethacin, Indoprofen, Ketoprofen, Ketorolac, Mofezolac, Naproxen, Nifenazone, Phenacetin, Propyphenazone, Sutrofen, Tenoxicam, Terofenamate, Tolfenamic acid, Tramadol, Viminol;

Androgens e.g. Boldenone, Cloxotestosterone, Mestanolone, Mesterolone, Methandrostenolone, Norethandrolone, Normethandrone, Oxandrolone, Oxymesterone, Oxymetholone, Prasterone, Stanolone, Stanozolol, Testosterone,

Angiotensin II receptor antagonists e.g. Candesartan, Eprosartan, Ibesartan, Losartan, Va1sartan:

Anorexics, e.g. Aminorex, Amphecloral, Anphetamine, Benzphetamine, Chlorphentermine, Clobenzorex, Clortermine, F enfluramine, Norpseudoephedrine, Pentorex, Phendimetrazine, Phenmetrazine;

Anthelmintics e.g. Arecoline, Aspidin, Aspidinol, Becanthone, Hycantone,.

Antiallergics e.g. Amlexanox, Astemizole, Azelastine, Cromolyn, Fempiprane, Ibudilast, Lodoxamide, Nedocromil, Oxatomide, Repirinast, Tazanolast, Hystamine, B Beclomethasone, Dexamethasone, Flunisolide, Fluticasone, Triamcinolone; Antialopecia agent. e.g. Cioteronel, Minoxidil

Antiamebics e.g. Arsthinol, Carbasone, Chlorbetamide, Ch1orphenoxamide, Emetine, fumaggilline, Iodoquinol, Verapamil

Antiarrhythmics e.g. Acebutol, Adenosine, Ajmaline, Alprenolol, Amiodarone, Atenolol, Bupranolol, Carazolol, Carteolol, Cloranolol, Indenolol, Ipratropium bromide, Lidocaine, Pindolol, Propafenone, Propranoll, Quinidine, Timolol, Verapamil;

Antiarteriosclerotic e.g. Pyridinol Carbamate;

Antiarthritics/Antirheumatics e.g. Actarit, Auranofin. Aurothioglucose, aurothioglicande, Azathioprine, Chloroquine, Gold sodium thiosulfate, Hydroxchloroquine, Methotrexate:

Antiasthmatics e.g. Azelastine, Cromolyn, Ibudilast, Ketotifen, Montelukast, Oxotomide, Pranlukast, Seratrodast, Zafirlukast, Zileuton, Beclomethasone, Budesonide, Dexamethasone, Flunisolide, Triamcinolon acetonide.

Antibacterials e.g. Amikacin, Gentamicin, KanamycinNeomicin, Tobramycin, Chloramphenicol, Thiamphenicol, Rifamide, Rifampin, Rifaximin, Cefaclor, Cefamandole Cefazolin, Cefiime, Cefazolin, Amoxicillin, Ampicillin, Oxacillin, Lindomycin, Erythromycin, Gramicidin, Teicoplanin, Vancomycin, Ch1ortetracycline Doxycylline, Tetracycline, Trimetoprim, Nifuradene, Nitrofurantoin, Ciprofloxacin, Ofloxacin, Lomefloxacin, Benzylsulamide, Chloraminet, Mafenide, Sulfabenzamide, Sulfacetamide, Sulfadiazine, Sulfadoxine, Sulfaguanidine, Sulfalene, Sulfanilamide, Sulfanylurea, sulfafazolel Sulfathiazole, Acedapsone, Dapsone, Solasu1fone, Ethinamide, Furonazide, Isoniazide, Streptomycin

Anticholinergics, e.g. Atropine, Fentomum bromide, Homatropine, Hyoscyamine, Ipratropium bromide, Isopropramide iodide, Scopolamine, Tropicamide:

Anticoagulants e.g. Acecumarol, Bromindione, Clorindione, Coumetarol, Dicumarol, Diphenadione, Fluindione, Heparin, Hirundin, Phenindione, Warfarin;

Anticonvulsants e.g. Albutoin, Aloxidone, Aminoglutethimide, Beclamide, Carbamazepine, Clonazepam, Ethadine, Ethotoin, Felbamate, Mephenytoin, Narcobarbital, Nimethazepam, Nitrazepam, Paramethadione, Phenacemide, Phenobarbital, Phenitoin, etc..

Antidepressant, i.e.: Citalopram, Fencaine, Nefopam, Iproclozide, Isocarboxazid, Nialamide, Rolyciprine, Maprotiline, Metra1indole, Amytriptiline, Clomipramide, Desipramide, Dibenzepin, Imipramide, Trimipramide, Bupropion, etc..

Antidiabetic, i.e.: Buformin, Phenformin, Insulin, Carbutamide, Chlorpopamide, Glipizide, Phenbutamide, Tolazamide, Tolbutamide, Tolcyclamide etc.

Antidiarreal, i.e.: Acetorphan, Catechin, Difenoxin, Diphenoxylate, Loperamide, Mebiquine, etc

Antidiuretic, i.e.: Desmopressin, Felypressin, Ornipressin, Vasopressin, etc..

Antidote, i.e.: Acetylcysteine, Cysteamine, Methionine, Folinic Acid, etc..

Antidyskinetic, i.e.: Amantidine, Clonidine, Haloperidol, Pimozide, Tetrabenazine etc..

Antiemetic, i.e. : Alizapride, Azasentron, Benzquinamide, Bromopride, Buclizine, Ch1orpromazine, Cyclizine, Domperidone, Granisetron, Meclizine, Metoclopramide, Ondansentron, Prochlorerazine, Scopolamine, Su1piride, Tropistron, etc..

Antifungal i.e.: Butenafine, Butoconazole, Econazole, Fenticonazole, Miconazole, Tolciclate, Tolindate, Fluconazole, Buclosamide, Triacetin, etc..

Antiglaucoma i.e.: Acetozolamide, Betaxolol, Bupranolol, etc..

Antigout i.e.: Allopurinol, Colchicine, Probenecid, Sulfipyrazone, etc.

Anthistaminic i.e.: Acrivastine, Brompheniramine, Chlorpheniramine, Dimethindene, Pheniramine, Tolpropamine, Clemastine, Diphenidramine, Medrilamyne, Cetirizine, Chlorcyclizine, Cinnarizine, Hidroxyzine, Fenethazine, Promethazine, Loratadine, Antazoline, Astemizole, Azelastine, Ebastine, Fexofenadine, Terfenadine, etc..

Anthyperlipoproteinemic i.e.. Cholestiramine, Benzofibrate, Clofibrate, Etofibrate, Genfibrozil, Atorvastatin, Lovastatin, Niceritrol, Thyroxine, Carnitine, Chondroitinsulfate, Ornithine, Probucol, etc..

Anthypertensive i.e.: Bufuralol, Acebutolol, Atenolol, Carteolol, Metoprolol, Moprolol, Pindolol, Propranolol, Timolol, Ch1orthiazide, Cyclopenthiazide, Hydroflumethazide, Benazepril, Captopril, Lisinopril, Ramipril, Amlodipine, Felodipine, Lacidipine, Nicardipine, Nitrendipine, Bethnide, Budralazine, Hydralazine; Pheniprazine, Phentolamine, Bunazosin, Prazosin, Reserpine, Furosemide, Ajma1ine, Fenoldopam, Mebutamate, Methildopa, Minoxidil, etc..

Antihypotensive i.e.: Dopamine, Etilefrin, Norepinephrine, Synephrine, etc..

Anti-inflammatry nonsteroidal i.e.: Etofenamate Flufenamic Acid, Mecoflenamic Acid, Tolfenamic Acid, Aceclofenac, Alclofenac, Bromfenac, Diclofenac Sodium, Etodolac, Ibufenac, Indomethacin, Pirazolac, Sulindac, Tolmetin, Fenbufen, Ketorolac, Alminoprofen, Fenoprofen, Flurbiprofen, Ibuprofen, Ketoprofen, Naproxen, Feprazone, Benorylate, Piroxicam, Bendazac, Nimesulide, etc..

Antimalarial i.e.; chloroquine, Chlorproquanil, Cinchonide, Cycloguanil, Quinidine, etc..

Antimigraine i.e.: Dolasetron, Ergocornine, Ergocriptyne, Ergot, Ergotamine, Lomerizine, Sumatriptan, etc..

Antiparkinsonian i.e.: Amantadine, Bromocriptine, Carbidopa, Levodopa, etc..

Antipsychotic i.e.: Alizapride, Amilsulpiride, Sulpiride, Risperidone, Ha1operidol, Acetophenazine, Chlorpromazine, Fluphenazine, Perazine, etc..

Antipyretic i.e.: Acetaminophen, Alclofenac, Aspirin, Benorilate, Indomethacin, etc..

Antispasmodic i.e.: Aminopromazine, Fentonium Bromide, Rociverine, Tiropramide, etc..

Antitussive ie.: Cloperastine, Codeine and derivetives, Dextromethorphan. Morclofone, etc..

Antiulcerative i.e.: Acetoxolone, Cimetidine, Famotidine, Omeprazole, Pirenzepine, Ranitidine, Sucralfate, etc..

Anxiolytic i.e.: Buspirone, Alprazolam, Broazepam, Camazepam, Lorazepam, Nordazepam, Meprobamate, etc..

Bronchodilator i.e.: Albuterol, Bambuterol, Calbiterol, Clenbuterol, Clorprenaline, Ephedrine, Ephineprine, Folmoterol, Metaproterenol, Salmeterol, Terbutaline, Ipratroprium Bromide, Teophilline and derivatives, etc..

Calcium channel blocker i.e.: Diltiazem, Verapamil, Amlodipine, Lacidipine, Micardipine, Nifedipine, Nomerizine, etc..

Cardiotonic i.e.: Digitalin, Digitoxin, Digoxin, Dopamine, Uabain, Scillaren, etc..

Choleretic i.e.: Cholic Acid, Cynerin, Dehydrocholic Acid, Dehoxycolic Acid, Taurocolic Acid, etc..

Cholinergic i.e.: Acetylcholine, Benzepirinium Bromide, Carbachol, Neostigmine, Physostigmine, etc..

CNS stimolant i.e.: Amphetamine, Caffeine, Fenozolone, Phentermine, etc..

Diuretic ie: Bendroflumethiazide, Benzylhytrochlorothiazide, Chlorothiazide, Indapamide, Mersalil, Candrenone, Oleandrin, Spironolattone, Acetazolamide, Butazolamide, Clopramide, Furosemide, Isosorbide, etc..

Dopamine receptor agonist i.e.: Bromocriptine, Cabercoline, Dopexamine, Fenoldopam, etc..

Dopamine receptor antagonist i.e.: Amisulpride, Domperidone, Metoclopamide, Sulpiride, etc..

Enzyme i.e.: Amylase, Lysozyme, Papain, etc..

Expetorant i.e.: Ambroxol, Bromhexine, Carbocysteine, Guaiacol, Guaifenesin, etc..

Gastric and Pancreatic secretion stimulant, i.e.: Carnitine Ceruletide etc..

Gastric proton pump inhibitor, i.e.: Lansoprazole, Omeprazole, Pantoprazole, etc..

Gastric secretion inhibitor, i.e.: Enterogastrone, Octretide, Telenzepine, etc..

Gastroprokinetic, i.e.: Cinitapride, Cisapride, Fenotozine, Loxiglumide, etc..

Glucocorticoid, i.e.: Beclomethasone, Bethometasone, Budesonide, Chloroprednisone, Clobetasone, Cortisone, Corticosterone, Deflazacort, Dexamethasone, Flumethasone, Fluocinolone Acetonide, Fluazacort, Fuorometholone, Flunisolide, Fluprednisolone, Hydrocortisone, Methylprednisolone, Prednisolone, Prednisone, Triamcinolone etc..

Hemolytic, i.e.: Pheni1hydrazine etc..

Histamine H₂-receptor antagonist, i.e.: Cimetidine, Ebrotidine, Famotidine, Nizatidine, Ranitidine, etc..

Laxative/Cathartic i.e.: Frangulin, Phenolphtaleine, Picosulfate sodium, etc..

Leukotriene antagonist, i.e.: Ibudilast, Montelukast, Pran1ukast, Zafirlukast etc..

Lipotropic, i.e.: Buserelin, Goserelin, Histrelin, Leuprolide, Nafarelin, Triptorelin etc..

Mineralcorticoid, i.e.: Aldosterone, Deoxycorticosterone, Fludrocortisone etc..

Monoamine oxidase inhibitor, i.e.: Iproniazid, Moclobemide, Phenoxypropazine, Selegeline, etc..

Mucolitic, i.e.: Acetylcysteine, Bromexine, Carbocysteine, Lysozime, Sobrerol, Tyloxapol, etc..

Muscle relaxant, i.e.: Afloqualone, Baclofen, Curare, Cyclarbamate, Dandrolene bromide, Diazepam, Eperisone, Flumetramide, Mephenesin Mephenaxolone, Methaxolone, Methocarbamol, Nimethazepam, Succyinylcholine Bromide Tetrazepam, Tubocurarine, etc..

Narcotic Antagonist. i.e.: Amiphenazole, Naloxone, Naltaxone etc..

Nootropic, i.e.: Aceglutamide, Besipiride, Piracetam, Vinconate, etc..

Oxytocic, i.e.: Carboprost, Deaminooxytocic, Ergonovine, Gemeprost, Methylergonovine, Oxytocin, Prostaglandin E², Prostaglandin F^{2a} etc..

Progestogen, i.e.: Drospirenone, Dydrogesterone, Ethynodiol, Flurogestone acetato, Lynestrenol, Medrogestone, Medroxyprogesterone, Megestrol acetato Norgesterone, Pentagestrone, Progesterone, etc..

Prolactin hinhibitor, i.e.: i.e: Bromocriptine, Cabergoline, Lisuride, Metergoline, Quinagoline, etc..

Prostaglandin/Prostaglandin analog, i.e.:

Beraprost, Carboprost, Enprostil, Gemeprost, Limaprost, Misoprostol, Prostacyclin, Prostaglandin E₁,E₂, F₂ₐ etc..

Respiratory stimulant, i.e.: Almitrine, Bemegride, Cropropamide, Dimorpholamine, Lobeline, Pyridopylline,etc.

Retroviral transcriptase inhibitor, i.e.: Delavirdine, Didanosine, Dideoxyadenosine, Lamivudine, Stavudine, Zidovudine

Sedative/Hypnotic, i.e: Accarbromal, Butoctamide, Di ethylbromoactamide, Niaprazine, Trimetozine, Zolpidem, Zopiclone, Allobarbital, Amobarbital, Barbital, Cyclopentobarbital, Hexobarbital, Mephobarbital, Narcobarbital, Pentobarbital, Phenobarbital, Tetrabarbital, Estazolam, Flunitrazepam, Flurazepam, Loprazolam, Lorttletazepam, Nitrazepam, Piperidione, Acetophenone, Clomethiazole Doxylamine, Temazepam, Triazolam, Methaqualone, Glutethimide, etc..

Serotonin Noradrenaline reuptake inhibitor, i.e.: Duloxetine, Velanfaxine,etc.

Serotonin reuptake agonist, i.e.: Buspirone, Eltoprazine, Ergotamine, Sumatriptan etc..

Serotonin receptor antagonist, i.e.: Azasentron, Dolasentron, Granisentron, Ondasentron, Ritanserin, Tropisentron, etc..

Serotonin uptake inhibitor, i.e.: Fomexitine, Fluoxetine, Paroxetine etc..

Vasodilator, i.e.: Cinnarizine, Citicoline, Fenoxedil, Flunarizine, Lomerizine, Nicergoline, Nimodipine, Papaverine, Amotriphene, Vincamine, Efloxate, Nitroglicerin, Pentrinitrol, Trapidil Bradykinin, Inositol, Nicergoline, Pentifillyne, Tlazoline,etc.

Vitamins, i.e.: Calcitriol, Ergosterol, Vitamin D, D₂, D₃, Ascorbic acid, β-Carotene, vitamin B₁₂ etc.

The solid pharmaceutical preparations of the present inventions may take various forms. They may be buccal tablets adapted to dissolve in the mouth. Such tablets may be placed in the buccal cavity, on the tongue or between the cheek and gums, for dissolution over a period depending on the chosen excipients. One preferred embodiment is a fast-melting buccal tablets made from a fluidised-bed granulated blend containing polyalcohol such as is disclosed in our WO-A-99/04758.

Other suitable embodiments include sublingual tablets, which are adapted to be placed under the tongue where the active ingredient can be absorbed directly into the blood stream through the mucosa.

A further embodiment is a chewable tablet. Techniques and materials for making chewable tablets are well known.

A further embodiment is a preparation adapted to be dissolved or dispersed in a carrier such as water for ingestion. Again, suitable excipients for these purposes are well known and it may be necessary only to include the necessary cyclodextrin in the preparation. For example, effervescent agents such as bicarbonates may be included in the formulation. The preparation may be in the form of tablets, granules or powder. The cyclodextrin for use according to the present invention may be an α, β, or γ-cyclodextrin. Besides the commonly available α, β, and γ-cyclodextrins, cyclodextrin derivatives such as hydroxypropyl-β-cyclodextrin, and acylated and modified cyclodextrins, for example those described in U.S. Pat. Nos. 5,654,422 and 5,633,368 (both to Hirsenkorn) and WO91/13100 (Australian Commercial Research and Development Ltd.), are also available for use in the invention. In a preferred embodiment the cyclodextrin is a β-cyclodextrin or derivative thereof.

Pharmaceutical formulations according to the present invention may be prepared by any suitable method which creates a homogeneous mixture of active ingredient and cyclodextrin without requiring any particular processes conditions to generate complexes between active and cyclodextrin. Mixtures may be prepared by simple dry blending, or by blending with a small quantity of water or other solvent to facilitate homogenisation. Actives may be granulated with cyclodextrin using a fluid bed granulator (preferred) or a granulating blender, using sufficient water or other suitable solvent to achieve a satisfactory granulation, but generally without such quantities of water as might give rise to significant complex formulation, e.g. by converting the mix to a paste or slurry.

Whatever the mode of preparation, in the light of our new discovery it is not necessary to take any measures to achieve complexation of the active ingredient by the cyclodextrin. Since complex formation is typically time-consuming and expensive this represents a simplification and corresponding possible economic advantage in the present procedures and products.

By way of explanation, there are a number of well recognised methods for forming cyclodextrin inclusion complexes: the solution method, the co-precipitation method, the neutralisation method, the slurry method, the kneading method and the grinding method, e.g. as summarised in T. Loftsson, Pharmaceutical Technology Europe, October 99 Vol.11(10). These generally involve prolonged and intensive mixing of the active with the cyclodextrin, often under carefully controlled conditions, and sometimes with application of heat, followed by isolation or purification of the complexes.

The skilled reader would appreciate that the presence of some complexed active ingredient is not generally actually detrimental in the eventual preparation. Rather, it is positively advantageous for processing reasons to simplify the process in such a way that complex formation is relatively unlikely to take place, or takes place to only a very limited extent or not at all. Thus, the presence of a minor amount of complex active ingredient does not take a composition outside the scope of the present invention. Nevertheless we have carried out thermal analysis of cyclodextrin/active blends prepared by simple granulations using minimal solvent. These analyses showed both components retaining their original melting points, with no appearance of different melting-point solids which would indicate complex formation. Thus, particular embodiments of the present invention are those in which the active ingredient is at least mostly, or essentially, or substantially entirely uncomplexed by the cyclodextrin. Any suitable method may be used for determining this situation, although as explained above it is not critical to the technical effectiveness of the cyclodextrin in taste masking.

Other pharmaceutical excipients may also be blended or granulated into the active-cyclodextrin mixture. The nature of these excipients will depend upon the required final form of the pharmaceutical. For example, for preparation of fast-melting formulations according to WO -A-99/04758, polyalcohol such as any one or more of xylitol, sorbitol, mannitol, maltitol, erythritol and lactitol may be included.

The preparation may contain an ingestible acid component, e.g. citric acid, typically up to 30 wt % of the total.

The preparations may contain effervescence agent selected from various acid and/or base components. Suitable acids include citric, tartaric, malic, fumaric, adipic, succinic and alginic acids. Acid salts and anhydrides may also be used.

Suitable bases include solid carbonates and bicarbonates.

Preferably the preparation contains not more than about 10 wt % of effervescent agent, but this may be varied in accordance with known practice.

The pharmaceutical compositions of the present invention may contain a single active ingredient or a plurality of active ingredients. Where more than one active ingredient has a taste which is required to be masked, the quantity of cyclodextrin can be adjusted appropriately.

Satisfactory taste masking can typically be achieved using a molar ratio of active or actives to cyclodextrin of between 0.9:1 and 1:25, preferably between 1:1 and 1:15.

Specific embodiments of the present invention are illustrated by the following examples.

### EXAMPLE 1

Fast melting tablets each containing 10 mg Dextromethorphan HBr and 2 mg Chlorpheniramine maleate were prepared as follows.

| | | |
|---|---|---|
| Dextromethorphan HBr | 10 | g |
| Chlorpheniramine maleate | 2 | g |
| Xylitol | 106 | g |
| Sorbitol | 325 | g |
| β-cyclodextrin | 300 | g |
| Citric acid | 8 | g |

were granulated together with just sufficient water for granulation, containing 10 g of PEG. To the dried granulate was added:

| | |
|---|---|
| Aspartame | 10 g |
| Magnesium stearate | 5 g |
| Vanilla flavour | 24 g |

This mixture was blended for 15 minutes to reach homogeneity, and the resulting mixture compressed on a rotating tabletting machine, with a toroidal punch of 13 mm diameter, to give tablets weighing 800 mg each.

The resulting tablets showed good mechanical characteristics and had a pleasant taste when dissolved in the buccal cavity.

### Comparison 1

Example 1 was repeated omitting the cyclodextrin and adjusting the total weight to 500 mg to achieve the same active dose. The tablets had good mechanical and dissolution characteristics but a very unpleasant strong bitter taste in the mouth.

### EXAMPLE 2

Fast melting tablets each containing 10 mg Dextromethorphan HBr, 2 mg Chlorpheniramine maleate and 6.67 mg Lysozyme HCl were prepared as follows.

| | | |
|---|---|---|
| Dextromethorphan HBr | 10 | g |
| Chlorpheniramine maleate | 2 | g |
| Xylitol | 106 | g |
| Sorbitol | 318.3 | g |
| Lysozyme HCl | 6.67 | g |
| β-cyclodextrin | 300 | g |
| Citric acid | 8 | g |

were granulated together with just sufficient water for granulation, containing 10 g of PEG. To the dried granulate was added:

| | |
|---|---|
| Aspartame | 10 g |
| Magnesium stearate | 5 g |
| Tangerine flavour | 24 g |

The mixture was blended for 15 minutes to reach homogeneity, and the resulting mixture compressed on a rotating tabletting machine with a toroidal punch of 13 mm diameter, to give tablets weighing 800 mg each.

The resulting tablets showed good mechanical characteristics and pleasant taste when dissolved in the buccal cavity.

### Comparison 2

Example 2 was repeated omitting the cyclodextrin, adjusting the tablet weight to 500 g to achieve the same active dose. Although good in mechanical and dissolution properties, the tablets had a very unpleasant bitter taste I the mouth.

### EXAMPLE 3

Chewable tablets each containing 10 mg Dextromethorphan HBr, 2 mg Chlorpheniramine maleate and 6.67 mg Lysozyme HCl were prepared as follows.

| | | |
|---|---|---|
| Dextromethorphan HBr | 10 | g |
| Chlorpheniramine maleate | 2 | g |
| Fructose | 70 | g |
| Maize starch | 70 | g |
| Sorbitol | 528.33 | g |
| Lysozyme HCl | 6.67 | g |
| β-cyclodextrin | 350 | g |
| Citric acid | 10 | g |

were granulated together with just sufficient water, for granulation, containing 10 g PVP K 25. To the dried granulate was added

| | |
|---|---|
| Aspartame | 14 g |
| Acesulfame K | 1 g |
| Magnesium stearate | 6 g |
| Orange flavour | 22 g |

The mixture was blended for 15 minutes to reach homogeneity, and the resulting mixture compressed on a rotating tabletting machine with a toroidal punch of 16 mm diameter, to give tablets weighing 1100 mg each.

The resulting tablets showed good mechanical characteristics and had a pleasant taste when chewed.

### Comparison 3

Example 3 was repeated omitting the cyclodextrin and adjusting the tablet weight to 750 mg to maintain the dose of active ingredients. The tablet had a very unpleasant strong bitter taste when chewed.

### EXAMPLE 4

Effervescent tablets each containing 10 mg Dextromethorphan HBr, 2 mg Chlorpheniramine maleate and 6.67 mg lysozyme HCl were prepared as follows:

| | | |
|---|---|---|
| Dextromethorphan HBr | 10 | g |
| Chlorpheniramine maleate | 2 | g |
| Lysozyme HCl | 6.67 | g |
| β-cyclodextrin | 350 | g |
| Citric acid | 1400 | g |

were granulated with just sufficient water for granulation, containing 18 g of β-carotene. To the resultant dry granulate was added

| | | |
|---|---|---|
| Sodium bicarbonate | 694 | g |
| Sorbitol | 911.3 | g |
| Aspartame | 20 | g |
| Acesulfame K | 8 | g |
| Orange flavour | 80 | g |

The mixture was dried for 20 minutes, and compressed on a rotating tabletting machine with a toroidal punch of 22 mm diameter, to give tablets weighing 3500 mg each.

The resulting tablets showed good mechanical characteristics and pleasant taste when dissolved in water and drunk.

### Comparison 4

Example 4 was repeated omitting the cyclodextrin and adjusting the tablet size to achieve the same dose. The tablets dissolved well but the solution had an unpleasant bitter taste.

### EXAMPLE 5

Fast melting tablets each containing 2 mg brompheniramine maleate and 5 mg phenylephrine HCl were prepared as follows:

Brompheniramine maleate 2 g

| | | |
|---|---|---|
| Phenylephrine HCl | 5 | g |
| Mannitol | 500 | g |
| Sorbitol | 349 | g |
| β-cyclodextrin | 199 | g |
| Citric acid | 22 | g |
| Crospovidine | 60 | g |

were granulated together in just sufficient water for granulation, containing 15 g of PEG. To the resultant dry granulate was added

| | |
|---|---|
| Aspartame | 10 g |
| Magnesium stearate | 8 g |
| Orange flavour | 30 g |

The mixture was blended for 15 minutes to reach homogeneity, and compressed on a rotating tabletting machine with a toroidal punch of 13 mm diameter, to give tablets weighing 1200 mg each.

The resulting tablets showed good mechanical characteristics and pleasant taste when dissolved in the buccal cavity.

### Comparison 5

Example 5 was repeated omitting the cyclodextrin and adjusting tablet size to 1001 mg to maintain the dose. The resulting tablets had good mechanical properties but an unpleasant bitter taste when dissolved in the mouth.

### EXAMPLE 6

Water soluble granulate was prepared as follows.

| | | |
|---|---|---|
| Ibuprofen | 200 | g |
| Sorbitol | 2706 | g |
| β-cyclodextrin | 1000 | g |
| Citric acid | 14 | g |

were granulated with just sufficient water. To the resultant dried granulate was added

| | |
|---|---|
| Aspartame | 20 g |
| Orange flavour | 60 g |

The mixture was blended for 15 minutes to reach homogeneity, and divided into 4000 mg doses packaged into aluminium paper sachets containing 200 mg ibuprofen each.

The resultant granulates had a pleasant taste when dissolved in water and drunk.

### Comparison 6

Example 6 was repeated omitting the cyclodextrin and reducing sachet contents to 3000 mg to maintain dose size. The dissolved granules had an unpleasant burning and irritating taste.

### EXAMPLE 7

Water soluble granulates containing 50 mg ketoprofen per dose were prepared as follows.

| | | |
|---|---|---|
| Ketoprofen | 60 | g |
| Saccharose | 787 | g |
| β-cyclodextrin | 1116 | g |
| Citric acid | 7 | g |
| Saccharine | 10 | g |
| Orange flavour | 30 | g |

were dry-blended at room temperature for 15 minutes to reach homogeneity. The resultant mixture was divided into 2000 mg doses packaged into aluminium paper sachets.

The resultant granulates had a pleasant taste when dispersed in water and drunk.

### Comparison 7

When the cyclodextrin was omitted from the Example 7 preparation the dispersed granulates had an unpleasant irritating and burning taste.

### EXAMPLE 8

Water soluble granulates containing 70 mg sumatriptan per dose were prepared as follows.

| | | |
|---|---|---|
| Sumatriptan | 70 | g |
| Saccharose | 1037 | g |
| β-cyclodextrin | 1345 | g |
| Citric acid | 8 | g |
| Saccharine | 10 | g |
| Orange flavour | 30 | g |

were simply blended for 15 minutes at room temperature to reach homogeneity. The resultant mixture was divided into 2500 mg doses packaged into aluminium paper sachets.

The resultant granulates exhibited a pleasant taste when dispersed in water and drunk.

### Comparison 8

Example 8 was repeated omitting the cyclodextrin, reducing the dose to 1155 g to maintain the active dose. Dispersed in water, the granulate had an unpleasant bitter taste.

### EXAMPLE 9

Fast melting tablets each containing 0.66 mg β-methasone disodium phosphate were prepared as follows.

| | | |
|---|---|---|
| β-methasone disodium phosphate | 0.66 | g |
| Mannitol | 42.5 | g |
| Sorbitol | 20 | g |
| Xylitol | 25.1 | g |
| β-cyclodextrin | 21 | g |
| Citric acid | 4 | g |

were granulated together in just sufficient water for granulation containing 15 g of PEG. To the resultant granulate was added

| | | |
|---|---|---|
| Aspartame | 1.25 | g |
| Magnesium stearate | 0.625 | g |
| Apple flavour | 3.6 | g |

The mixture was blended for 15 minutes to reach homogeneity, and compressed on a rotating tabletting machine with a toroidal punch of 8 mm diameter, to give tablets weighing 120 mg each.

The resulting tablets showed good mechanical characteristics and pleasant taste when dissolved in the buccal cavity.

### Comparison 9

When Example 9 was repeated without the cyclodextrin, reducing the tablet weight to 99 mg to maintain the active content, the resulting tablet had good mechanical properties but an unpleasant and very bitter taste when dissolved in the mouth.

### EXAMPLE 10

Fast melting tablets each containing 2 mg brompheniramine maleate, 5 mg phenylephrine HCl, and 10 mg dextromethorphan were prepared as follows.

| | | |
|---|---|---|
| Brompheniramine maleate | 2 | g |
| Phenylephrine HCl | 5 | g |
| Dextromethorphan HCl | 10 | g |
| Mannitol | 450 | g |
| Sorbitol | 176 | g |
| β-cyclodextrin | 375 | g |
| Citric acid | 12 | g |

were granulated together with just sufficient water. To the resultant granulate was added

| | | |
|---|---|---|
| Aspartame | 10 | g |
| Magnesium stearate | 10 | g |
| Apple flavour | 30 | g |
| Crospovidine | 120 | g |

The mixture was blended for 15 minutes to reach homogeneity, and compressed on a rotating tabletting machine with a toroidal punch of 8 mm diameter, to give tablets weighing 1200 mg each.

The resulting tablets showed good mechanical characteristics, and a pleasant taste when dissolved in the buccal cavity.

### Comparison 10

Example 10 was repeated omitting the cyclodextrin and with the tablets at 825 mg to give the same active dose. The resulting tablets had good mechanical properties but an unpleasant and persistent bitter taste when dissolved in the mouth.

### Comparison 11

Fast-melting tablets, each containing 100 mg of aceclofenac, were prepared as follows.

| | | |
|---|---|---|
| Aceclofenac | 100 | g |
| Mannitol | 320 | g |
| Sorbitol | 154 | g |
| Xylitol | 195 | g |
| β-cyclodextrin | 481 | g |
| Citric acid | 18 | g |

were granulated together with just sufficient water for granulation, containing 15 g of PEG. To the resultant dried granulate was added

| | |
|---|---|
| Aspartame | 41 g |
| Magnesium stearate | 6 g |
| Glycirrhiza and Alpine flavouring | 55 g |

The mixture was blended for 15 minutes to homogeneity and compressed to 1385 mg tablets on a rotating tableting machine using a 16 mm-diameter toroidal punch.

The tablets had good mechanical properties, and a pleasant taste when dissolved in the buccal cavity.

### Comparison 11

Example 11 was repeated omitting the cyclodextrin, and producing 842 mg tablets (with a 13 mm punch) to achieve the same 100 mg dose of aceclofenac.

The resulting tablets had good mechanical properties but an unpleasant and persistent bitter taste when dissolved in the buccal cavity.

1. A solid oral pharmaceutical preparation, adapted for oral administration by dispersion of pharmaceutically active ingredient from the solid preparation in the mouth, or by drinking an aqueous dispersion of the solid preparation;
the preparation comprising a blend of said active ingredient with cyclodextrin as taste-masking agent for the active ingredient which is not complexed thereby.

2. A solid oral pharmaceutical preparation according to claim 1 in the form of a tablet.

3. A solid oral pharmaceutical preparation according to claim 2 in which the tablet is formulated as a solid buccal tablet, as a soluble sub-lingual tablet or as a chewable tablet.

4. A solid oral pharmaceutical preparation according to claim 1 in the form of a granulate or dry blend.

5. A solid oral pharmaceutical preparation according to any one of the preceding claims which contains polyalcohol blended with the cyclodextrin and active ingredient.

6. A solid oral pharmaceutical preparation according to any one of the preceding claims which contains ingestible organic acid or acid salt blended with the cyclodextrin and active ingredient.

7. A solid oral pharmaceutical preparation according to any one of the preceding claims which contains flavour and/or sweetener.

8. A solid oral pharmaceutical preparation according to any one of the preceding claims which contains an effervescence agent.

9. A solid oral pharmaceutical preparation according to any one of the preceding claims in which the molar ratio of the active ingredient to cyclodextrin is between 0.9:1 and 1:25.

10. A solid oral pharmaceutical preparation according to any one of the preceding claims in which the cyclodextrin is β-cyclodextrin or derivative thereof.

11. A process of preparing a solid oral pharmaceutical preparation, adapted for oral administration by dispersion of pharmaceutically-active ingredient from the solid preparation in the mouth, or by drinking an aqueous dispersion of the solid preparation;
the process comprising blending the pharmaceutically-active ingredient with cyclodextrin to produce said preparation as a mixture containing the active ingredient substantially uncomplexed by the cyclodextrin.

12. A process according to claim 11 in which the mixture of cyclodextrin and active ingredient is granulated with sufficient liquid for granulation but insufficient liquid to convert the mixture to a paste.

13. A process according to claim 11 comprising compressing the granulate to form tablets.

14. A process according to any one of claims 11 to 13 including dry-mixing the cyclodextrin and active ingredient at ambient temperature or without heating.

15. A process according to any one of claims 11 to 14 in which the resulting preparation is in accordance with any one of claims 2 to 10.

16. The use, in the oral administration of a solid pharmaceutical preparation by dispersion of pharmaceutically-active ingredient from the solid preparation in the mouth, or by drinking an aqueous dispersion of the solid preparation, of cyclodextrin blended but not complexed with the active ingredient in the preparation to mask the taste of the uncomplexed active ingredient.

17. The use, in the preparation of a solid pharmaceutical preparation adapted for oral administration by dispersion of pharmaceutically-active ingredient from the solid preparation in the mouth, or by drinking an aqueous dispersion of the solid preparation, of cyclodextrin mixed with the active ingredient to mask the taste of the active ingredient which is not complexed with the cyclodextrin.
